(19) United States
(12) Patent Application Publication
(10) Pub. No.: US 2022/0296490 A1
(43) Pub. Date: Sep. 22, 2022

(54) Oral Care Products and Methods

(71) Applicants: Colgate-Palmolive Company, New York, NY (US); BASF SE, Ludwigshafen, NY (US)

(72) Inventors: Dennis ZHANG, Piscataway, NJ (US); Luciana RINAUDI MARRON, Somerset, NJ (US); Stacey LAVENDER, Chesterfield, NJ (US); Thomas SUBKOWSKI, Schriesheim (DE); Nina SCHNEIDER, Edingen-Neckarhausen (DE); Volker WENDEL, Seeheim-Jugenheim (DE); Claus BOLLSCHWEILER, Heidelberg (DE)

(21) Appl. No.: 17/831,796

(22) Filed: Jun. 3, 2022

Related U.S. Application Data
(63) Continuation of application No. PCT/US2020/063407, filed on Dec. 4, 2020.
(60) Provisional application No. 62/944,879, filed on Dec. 6, 2019.
(60) Provisional application No. 62/944,828, filed on Dec. 6, 2019.

(51) Int. Cl. A61K 8/64; A61K 8/21; A61Q 11/00

(52) U.S. Cl. CPC A61K 8/645; A61K 8/21; A61Q 11/00; A61K 2800/30

(57) ABSTRACT

Provided herein are oral care compositions comprising partially hydrolyzed plant protein, which are useful in methods of repairing or inhibiting dental erosion, promoting dental remineralization, and/or enhancing the anti-cavity effects of fluoride.

### CROSS REFERENCE TO RELATED APPLICATIONS

This application claims the benefit and priority of U.S. provisional application 62/944,828, filed on Dec. 6, 2019, and U.S. provisional application 62/944,879, filed on Dec. 6, 2019, the contents of both of which are hereby incorporated by reference in their entireties.

### BACKGROUND

Dental enamel is a thin, hard layer of calcified material that covers the crown of teeth. The major mineral component of dental enamel is hydroxyapatite, a crystalline form of calcium phosphate. Chemical erosion of dental enamel may arise from tooth exposure to acidic food and drinks or to stomach acids arising from gastric reflux. The erosion of dental enamel can lead to enhanced tooth sensitivity due to increased exposure of the dentin tubules and increased dentin visibility leading to the appearance of more yellow teeth. The salivary pellicle (a thin layer of salivary glycoproteins deposited on teeth) is integral in protecting the teeth against an erosive challenge. As a result, people that experience xerostomia are more susceptible to acid erosion damage.

Acids are also generated in the oral cavity when plaque containing cariogenic bacteria metabolize carbohydrates. Since plaque forms a barrier controlling the kinetics of proton and mineral diffusion through the enamel, plaque acids cause carious lesions. Incorporating fluoride ions in dentifrice formulations is the most common method to mitigate the effects of plaque acids. Fluoride reduces the rate of demineralization and enhances remineralization. Several approaches have also been developed to stabilize calcium phosphate salts or control the plaque pH to enhance remineralization.

Although methods have been developed to mitigate the effects of non-bacteria and bacteria generated acids on the teeth, there is still the need to provide improved oral care compositions that effectively repair the enamel from the effects of acid erosion and bacteria acids.

### BRIEF SUMMARY

The present inventors have unexpectedly found that partially hydrolyzed plant proteins, e.g., partially hydrolyzed proteins from wheat, rice, almond, potato, soya, pea or combinations thereof, for example partially hydrolyzed cereal proteins (hydrolyzed proteins from grains of the family Poaceae, for example partially hydrolyzed wheat protein or partially hydrolyzed rice protein, are effective in repairing or mitigating the effects of dental erosion, promoting dental remineralization, and enhancing the anti-cavity effects of fluoride. Typically, the compositions of the present invention utilize concentrations of hydrolyzed plant proteins in oral care compositions at concentrations above 3% by wt. of the total composition. Previously, using concentrations at such levels, was believed to be costly and could potentially involve unknown safety concerns. However, despite these obstacles, the inventors have now found that oral care formulations, with concentrations of certain hydrolyzed plant protein above 3% by wt. of the composition, can be both safe and efficacious. The present inventors have also found, that formulations with concentrations of hydrolyzed plant protein above 3% by wt. of the composition, have remineralization properties equal to, or better, than certain oral care formulations containing fluoride.

For example, in one embodiment, partially hydrolyzed plant proteins comprising oligo- and polypeptide molecules having a molecular weight distribution of about 500 D to about 10000 D, e.g. 1000 D to 5000 D are prepared for formulation with ingredients of a suitable orally acceptable carrier, by diluting in buffer, e.g., a phosphate buffer such as Na2HPO4 buffer (1.5 mM) and CaCl₂) (2.5 mM), to provide a buffered solution having a desired pH, e.g. pH 6-8, e.g., pH 7-8, e.g., about pH 7.5, filtering and centrifuging the solution to obtain a filtrate comprising the partially hydrolyzed plant protein. A biocide (for example cetylpyridinium chloride at 0.1%) and fluoride may be added to the filtrate. The partially hydrolyzed plant protein may then be combined with components of an orally acceptable carrier, for example a toothpaste or mouthwash base, to provide an oral care composition for repairing or mitigating the effects of dental erosion, promoting dental remineralization, and enhancing the anti-cavity effects of fluoride.

In one aspect, this present invention relates to an oral care composition (Composition 1), e.g., a dentifrice or an oral gel, comprising:
a) partially hydrolyzed plant protein, wherein the partially hydrolyzed plant protein comprises oligo- and polypeptide molecules having a molecular weight distribution of about 500 D to about 10000 D, e.g. 1000 D to 5000 D, for example 500D to 10000 D or 1000 D to 5000 D; for example having an average molecular weight of 1000 D to 5000 D;
b) an orally acceptable carrier,
wherein the partially hydrolyzed plant protein is present in the composition in an amount of from 3.05 weight % to 10.5 weight % by total weight of the composition, e.g., 3.5 weight %-10.5 weight % by total weight of the composition, e.g., 3.5 weight %-10.5% weight % by total weight of the composition, e.g., 3.5 weight %-4.5% weight % by total weight of the composition, e.g., 4.5 weight %-5.5% weight % by total weight of the composition, e.g., 9.5 weight %-10.5% weight % by total weight of the composition, e.g., e.g., 5.5 weight %-10.5 weight % by total weight of the composition, about 4 weight %, or about 5 weight %, about 6 weight %, about 7 weight %, about 8 weight %, about 9 weight %, or about 10 weight % by total weight of the composition. For example, the disclosure provides:
1.1. Composition 1 wherein the partially hydrolyzed plant protein is obtained from grains of the family Poaceae, e.g., is partially hydrolyzed maize (corn), wheat, rice, barley, oat, or millet protein.
1.2. Composition 1.1 wherein the partially hydrolyzed plant protein is partially hydrolyzed wheat protein or hydrolyzed rice protein.
1.3. Composition 1 wherein the partially hydrolyzed plant protein is obtained from wheat, rice, almond, potato, pea, soya or combinations thereof.
1.4. Any foregoing Composition wherein the oral care composition comprises fluoride.
1.5. Any foregoing Composition wherein the oral care composition has been adjusted to a desired pH, e.g., pH 6-8, e.g., pH 7-8, e.g, by using a phosphate buffer.
1.6. Any foregoing Composition wherein the oral care composition comprises a biocide, e.g., cetylpyridinium chloride (CPC) at an effective concentration, e.g., 0.1% by weight of the filtrate.
1.7. Any foregoing Composition wherein the partially hydrolyzed plant protein (e.g., hydrolyzed wheat protein) is present in the composition in an amount of from 3.05 weight % to 10.5 weight % by total weight of the composition, e.g., 3.5 weight %-10 weight % by total weight of the composition, e.g., 3.5 weight %-10% weight % by total weight of the composition, e.g., 3.5 weight %-4.5% weight % by total weight of the composition, e.g., 4.5 weight %-5.5% weight % by total weight of the composition, e.g., 9.5 weight %-10.5% weight % by total weight of the composition, e.g., e.g., 5.5 weight %-10.5 weight % by total weight of the composition, about 4 weight %, or about 5 weight %, about 6 weight %, about 7 weight %, about 8 weight %, about 9 weight %, or about 10 weight % by total weight of the composition.
1.8. Any of the forgoing compositions wherein the partially hydrolyzed plant protein (e.g., hydrolyzed wheat protein) is present in the composition in an amount of from 3.05 weight % to 30.5 weight % (e.g., about 30 weight %) by total weight of the composition.
1.9. The Composition of 1.7, wherein the partially hydrolyzed plant protein (e.g., hydrolyzed wheat protein) is present in the composition from 3.5 weight %-4.5% weight % (e.g., about 4 weight %) by total weight of the composition.
1.10. The Composition of 1.7, wherein the partially hydrolyzed plant protein (e.g., hydrolyzed wheat protein) is present in the composition at about 4 weight %, by total weight of the composition.
1.11. The Composition of 1.7, wherein the partially hydrolyzed plant protein (e.g., hydrolyzed wheat protein) is present in the composition from 4.5 weight %-5.5% weight % (e.g., about 5 weight %) by total weight of the composition.
1.12. The Composition of 1.7, wherein the partially hydrolyzed plant protein (e.g., hydrolyzed wheat protein) is present in the composition from 9.5 weight %-10.5% weight % (e.g., about 10 weight %) by total weight of the composition.
1.13. The Composition of 1.7, wherein the partially hydrolyzed plant protein (e.g., hydrolyzed wheat protein) is present in the composition from 5.5 weight %-10.5% weight % (e.g., about 6 weight %, about 7 weight %, about 8 weight %, or about 9 weight %) by total weight of the composition.
1.14. The composition of 1.8, wherein the partially hydrolyzed plant protein (e.g., hydrolyzed wheat protein) is present in the composition from 10.5 weight %-20.5 wt % (e.g., about 15 wt %, about 20 wt %), or from 20.5 wt % to 30.5 wt % (e.g., about 25 wt %, about 30 wt %), by total weight of the composition.
1.15. Any foregoing composition wherein the Composition comprises an effective amount of fluoride.
1.16. Any foregoing Composition which comprises an amount of fluoride of 100 ppm to 2500 ppm, for example, 250 ppm to 750 ppm, for example about 500 ppm fluoride.
1.17. Any foregoing Composition which comprises an amount of fluoride of 2500 ppm to 5000 ppm.
1.18. Any foregoing Composition comprising an orally acceptable zinc salt or oxide, for example selected from zinc oxide, zinc citrate, zinc lactate, zinc phosphate, zinc acetate, zinc chloride, zinc complexes with amino acids, and mixtures of any of the foregoing, for example wherein the amount of zinc is from 0.1 weight % to 3 weight %, e.g., about 1 to about 2 weight %, calculated by weight of zinc ion.
1.19. Any foregoing Composition comprising an orally acceptable stannous salt, for example stannous fluoride or stannous chloride.
1.20. Any foregoing Composition wherein the composition is in a form selected from a mouthrinse, a toothpaste, a tooth gel, a tooth powder, a non-abrasive gel, a mousse, a foam, a mouth spray, chewing gum, and a tablet, for example dentifrice, e.g., a toothpaste or mouthrinse.
1.21. Any of the foregoing compositions, wherein the composition is in the form of a gel (e.g., wherein the gel is used for in-office or professional applications).
1.22. Any foregoing Composition, wherein the composition further comprises one or more agents selected from: abrasives, pH modifying agents, surfactants, foam modulators, thickening agents, viscosity modifiers, humectants, anti-calculus or tartar control agents, sweeteners, flavorants and colorants.
1.23. Any foregoing Composition wherein the composition is a toothpaste.
1.24. Any foregoing Composition comprising one or more soluble phosphate salts, for example wherein by “soluble phosphate salts” is meant an orally acceptable phosphate salt having a solubility in water of at least 1 g/100 ml at 25° C.; for example wherein the one or more soluble phosphate salts are sodium and/or potassium salts of pyrophosphates and/or polyphosphates, e.g., tripolyphosphates; e.g., wherein the one or more soluble phosphate salts comprise tetrasodium pyrophosphate (TSPP), sodium tripolyphosphate (STPP) or a combination of TSPP and STPP; for example, whereon the one or more soluble phosphate salts are present in an amount of 1-20%, e.g., 2-8%, e.g., ca. 5%, by weight of the composition.
1.25. Any foregoing Composition wherein the fluoride is provided by a salt selected from stannous fluoride, sodium fluoride, potassium fluoride, sodium monofluorophosphate, sodium fluorosilicate, ammonium fluorosilicate, amine fluoride (e.g., N′-octadecyltrimethylendiamine-N,N,N′-tris(2-ethanol)-dihydrofluoride), ammonium fluoride, titanium fluoride, hexafluorosulfate, and combinations thereof.
1.26. Any foregoing Composition which is a dentifrice comprising a humectant, e.g., selected from glycerin, sorbitol, propylene glycol, polyethylene glycol, xylitol, and mixtures thereof, e.g. comprising at least 30%, e.g., 40-50% glycerin, by weight of the composition.
1.27. Any foregoing Composition which is a dentifrice comprising one or more surfactants, e.g., selected from anionic, cationic, zwitterionic, and nonionic surfactants, and mixtures thereof, e.g., wherein the dentifrice base comprises an anionic surfactant, e.g., a surfactant selected from sodium lauryl sulfate, sodium ether lauryl sulfate, and mixtures thereof, e.g. in an amount of from about 0.3% to about 4.5% by weight, e.g. 1-2% sodium lauryl sulfate (SLS) by weight of the composition.
1.28. Any foregoing Composition which is a dentifrice comprising a zwitterionic surfactant, for example a betaine surfactant, for example cocamidopropylbetaine, e.g. in an amount of from about 0.1% to about 4.5% by weight, e.g. 0.5-2% cocamidopropylbetaine by weight of the composition
1.29. Any foregoing Composition which is a dentifrice comprising a viscosity modifying amount of one or more of polysaccharide gums, for example xanthan gum or carrageenan, silica thickener, and combinations thereof.
1.30. Any foregoing Composition which is a dentifrice comprising gum strips or fragments.
1.31. Any foregoing Composition which is a chewing gum.
1.32. Any foregoing Composition comprising flavoring, fragrance and/or coloring.
1.33. Any foregoing Composition which is a dentifrice comprising an effective amount of one or more antibacterial agents, for example comprising an antibacterial agent selected from: herbal extracts and essential oils (e.g., rosemary extract, tea extract, magnolia extract, thymol, menthol, eucalyptol, geraniol, carvacrol, citral, hinokitol, catechol, methyl salicylate, epigallocatechin gallate, epigallocatechin, gallic acid, miswak extract, sea-buckthorn extract), bisguanide antiseptics (e.g., chlorhexidine, alexidine or octenidine), quaternary ammonium compounds (e.g., cetylpyridinium chloride (CPC), benzalkonium chloride, tetradecylpyridinium chloride (TPC), N-tetradecyl-4-ethylpyridinium chloride (TDEPC)), phenolic antiseptics, hexetidine, octenidine, sanguinarine, povidone iodine, delmopinol, salifluor, metal ions (e.g., zinc salts, for example, zinc citrate, stannous salts, copper salts, iron salts), sanguinarine, propolis and oxygenating agents (e.g., hydrogen peroxide, buffered sodium peroxyborate or peroxycarbonate), phthalic acid and its salts, monoperthalic acid and its salts and esters, ascorbyl stearate, oleoyl sarcosine, alkyl sulfate, dioctyl sulfosuccinate, salicylanilide, domiphen bromide, delmopinol, octapinol and other piperidino derivatives, nicin preparations, chlorite salts; and mixtures of any of the foregoing; e.g., comprising cetylpyridinium chloride.
1.34. Any foregoing Composition which is a dentifrice comprising a whitening agent, e.g., a selected from the group consisting of peroxides, metal chlorites, perborates, percarbonates, peroxyacids, hypochlorites, and combinations thereof; e.g. hydrogen peroxide or a hydrogen peroxide source, e.g., urea peroxide or a peroxide salt or complex (e.g., such as peroxyphosphate, peroxycarbonate, perborate, peroxysilicate, or persulphate salts; for example calcium peroxyphosphate, sodium perborate, sodium carbonate peroxide, sodium peroxyphosphate, and potassium persulfate).
1.35. Any foregoing Composition which is a dentifrice comprising an agent that interferes with or prevents bacterial attachment, e.g., solbrol or chitosan.
1.36. Any foregoing Composition which is a dentifrice comprising a soluble calcium salt, e.g., selected from calcium sulfate, calcium chloride, calcium nitrate, calcium acetate, calcium lactate, and combinations thereof.
1.37. Any foregoing Composition which is a dentifrice comprising a physiologically or orally acceptable potassium salt, e.g., potassium nitrate or potassium chloride, in an amount effective to reduce dentinal sensitivity.
1.38. Any foregoing Composition which is a dentifrice comprising an anionic polymer, e.g., a synthetic anionic polymeric polycarboxylate, e.g., wherein the anionic polymer is selected from 1:4 to 4:1 copolymers of maleic anhydride or acid with another polymerizable ethylenically unsaturated monomer; e.g., wherein the anionic polymer is a methyl vinyl ether/maleic anhydride (PVM/MA) copolymer having an average molecular weight (M.W.) of about 30,000 to about 1,000,000, e.g. about 300,000 to about 800,000, e.g., wherein the anionic polymer is about 1-5%, e.g., about 2%, of the weight of the composition.
1.39. Any foregoing Composition which is a dentifrice comprising a breath freshener, fragrance or flavoring.
1.40. Any of the foregoing Compositions, wherein the pH of the composition is approximately neutral, e.g., pH 6-8, e.g., about pH 7.
1.41. Any of the foregoing Compositions, wherein the composition is free of any fluoride source (e.g., the composition does not contain a source of fluoride)
1.42. The composition of 1.41, wherein the composition contains from 3.5%-10.5% hydrolyzed wheat protein by weight of the composition (e.g., about 4%, about 5%, about 10%, or about 30% hydrolyzed wheat protein, by weight of the composition.)
1.43. The composition of 1.41, wherein the composition contains about 4% hydrolyzed wheat protein, by weight of the composition. (e.g., where the composition is in the form of a gel).
1.44. Any forgoing Composition, wherein the composition comprises a thickening agent, and wherein the thickening agent is selected from the group consisting of: carbomers (carboxyvinyl polymers), carrageenans, cellulosic polymers such as hydroxyethylcellulose, carboxymethylcellulose (CMC) and salts thereof (e.g., sodium carboxymethylcellulose), karaya, xanthan, gum arabic and tragacanth, colloidal magnesium aluminum silicate, and colloidal silica, acrylic acid (e.g., polyacrylic acid, e.g., Carbopol®).
1.45. Any of the forgoing compositions for use to reduce and inhibit acid erosion, clean the teeth, reduce bacterially-generated biofilm and plaque, reduce gingivitis, inhibit tooth decay and formation of cavities, and reduce dentinal hypersensitivity.
1.46. Any foregoing Composition for use in reducing, inhibiting or repairing dental enamel erosion.
1.47. Any foregoing composition for use in promoting remineralization of dental enamel.
1.48. Any foregoing composition for use in enhancing the anti-cavity effects of fluoride.

In still a further aspect, Composition 1 is a dentifrice comprising
a) a partially hydrolyzed wheat protein, comprising oligo- and polypeptide molecules having a molecular weight distribution of about 500 D to about 10,000 D, e.g., about 1000 D to about 5000 D; for example 500D to 10000 D, or 1000 D to 5000 D; for example having an average molecular weight of 1000 D to 5000 D; for example wherein the amount of hydrolyzed wheat protein 3.05 weight % to 10.5 weight % by total weight of the composition, e.g., 3.5 weight %-10.5 weight % by total weight of the composition, e.g., 3.5 weight %-10% weight % by total weight of the composition, e.g., 3.5 weight %-4.5% weight % by total weight of the composition, e.g., 4.5 weight %-5.5% weight % by total weight of the composition, e.g., 9.5 weight %-10.5% weight % by total weight of the composition, e.g., e.g., 5.5 weight %-10.5 weight % by total weight of the composition, about 4 weight %, or about 5 weight %, or about 6 weight %, or about 7 weight %, or about 8 weight %, or about 9 weight %, or about 10 weight % by total weight of the composition;
b) optionally an effective amount of fluoride; for example wherein fluoride is present in an amount of 100 ppm to 1500 ppm, for example, about 500 ppm; and
c) an orally acceptable carrier.

In yet another aspect, Composition 1 is an oral gel comprising
a) a partially hydrolyzed wheat protein, comprising oligo- and polypeptide molecules having a molecular weight distribution of about 500 D to about 10,000 D, e.g., about 1000 D to about 5000 D; for example 500D to 10000 D, or 1000 D to 5000 D; for example having an average molecular weight of 1000 D to 5000 D; for example wherein the amount of hydrolyzed wheat protein, e.g., 3.5 weight %-10 weight % by total weight of the composition, e.g., about 4 weight %, or about 5 weight %, or about 6 weight %, or about 7 weight %, or about 8 weight %, or about 9 weight %, or about 10 weight % by total weight of the composition;
b) optionally an effective amount of fluoride; for example wherein fluoride is present in an amount of 100 ppm to 1500 ppm, for example, about 500 ppm; and
c) an orally acceptable carrier.

In still a further aspect, Composition 1 is a dentifrice comprising
a) a partially hydrolyzed wheat protein, comprising oligo- and polypeptide molecules having a molecular weight distribution of about 500 D to about 10,000 D, e.g., about 1000 D to about 5000 D; for example 500D to 10000 D, or 1000 D to 5000 D; for example having an average molecular weight of 1000 D to 5000 D; for example wherein the amount of hydrolyzed wheat protein is about 4 weight %, by total weight of the composition;
b) optionally an effective amount of fluoride; for example wherein fluoride is present in an amount of 100 ppm to 1500 ppm, for example, about 500 ppm; and
c) an orally acceptable carrier.

In one aspect, the disclosure provides any of Compositions 1, et seq. for use in repairing or inhibiting dental erosion, promoting remineralization, and/or enhancing the anti-cavity effects of fluoride; for example for use in any of the following methods according to Method 1, et seq.

In another aspect, the disclosure provides a method (Method 1) of repairing or inhibiting dental erosion, promoting dental remineralization, and/or enhancing the anti-cavity effects of fluoride comprising applying to the teeth a composition, e.g., any of Composition 1, et seq. for example an oral care composition comprising:
a) partially hydrolyzed plant protein, comprising oligo- and polypeptide molecules having a molecular weight distribution of about 500 to about 10000 D, e.g. about 1000 to about 5000 D;
b) an orally acceptable carrier,
   wherein the partially hydrolyzed plant protein (e.g., hydrolyzed wheat protein) is present in the composition in an amount of from e.g., 3.5 weight %-10 weight % by total weight of the composition, e.g., 3.5 weight %-10% weight % by total weight of the composition, e.g., 3.5 weight %-4.5% weight % by total weight of the composition, e.g., 4.5 weight %-5.5% weight % by total weight of the composition, e.g., 9.5 weight %-10.5% weight % by total weight of the composition, e.g., 5.5 weight %-10.5 weight % by total weight of the composition, about 4 weight %, or about 5 weight %, about 6 weight %, about 7 weight %, about 8 weight %, about 9 weight %, or about 10 weight % by total weight of the composition, or in an amount from e.g., 10.5 weight %-30.5 weight % by total weight of the composition. For example, the disclosure provides:
1.1. Method 1 wherein the partially hydrolyzed plant protein is obtained from wheat, rice, almond, potato, pea, soya or combinations thereof; for example from grains of the family Poaceae, e.g., is partially hydrolyzed maize (corn), wheat, rice, barley, oat, or millet protein.
1.2. Method 1.1 wherein the partially hydrolyzed plant protein is partially hydrolyzed wheat protein or hydrolyzed rice protein.
1.3. Method 1 wherein the partially hydrolyzed plant protein is obtained from almond, potato or soya.
1.4. Any foregoing Method wherein the oral care composition comprises fluoride.
1.5. Any foregoing Method wherein the oral care composition has been adjusted to the desired pH, e.g., pH 6-8, for example pH 7-8, e.g., using a phosphate buffer.
1.6. Any foregoing Method wherein the oral care composition comprises a biocide, e.g., cetylpyridinium chloride (CPC) at an effective concentration, e.g., 0.1% by weight of the filtrate.
1.7. Any foregoing Method wherein the partially hydrolyzed plant protein (e.g., hydrolyzed wheat protein) is present in the composition in an amount of 3.1 weight % to 10 weight % by total weight of the composition, e.g., 3.5 weight %-4.5% weight % by total weight of the composition, e.g., 4.5 weight %-5.5% weight % by total weight of the composition, e.g., 9.5 weight %-10.5% weight % by total weight of the composition, e.g., e.g., 5.5 weight %-10.5 weight % by total weight of the composition.
1.8. The Method of 1.7, wherein the partially hydrolyzed plant protein (e.g., hydrolyzed wheat protein) is present in the composition in an amount of 3.5 weight %-4.5% weight % by total weight of the composition (e.g., about 4 weight %).
1.9. The Method of 1.7, wherein the partially hydrolyzed plant protein (e.g., hydrolyzed wheat protein) is present in the composition in an amount of 4.5 weight %-5.5% weight % by total weight of the composition (e.g., about 5 weight %).
1.10. The Method of 1.7, wherein the partially hydrolyzed plant protein (e.g., hydrolyzed wheat protein) is present in the composition in an amount of 9.5 weight %-10.5% weight % by total weight of the composition (e.g., about 10 weight %).
1.11. The Method of 1.7, wherein the partially hydrolyzed plant protein (e.g., hydrolyzed wheat protein) is present in the composition in an amount of 5.5 weight %-10.5% weight % by total weight of the composition (e.g., about 6 weight %, about 7 weight %, about 8 weight %, about 9 weight %).
1.12. Any foregoing composition wherein the Composition comprises an effective amount of fluoride.
1.13. Any foregoing Method wherein the amount of fluoride is 100 ppm to 1000 ppm, for example, about 500 ppm fluoride.
1.14. Any foregoing Method wherein the composition is in a form selected from a mouthrinse, a toothpaste, a tooth gel, a tooth powder, a non-abrasive gel, a mousse, a foam, a mouth spray, and a tablet.
1.15. Any forgoing Method, wherein the composition is in the form of an oral gel (e.g., an oral gel meant for office or professional use).
1.16. Any foregoing Method, wherein the composition further comprises one or more agents selected from: abrasives, pH modifying agents, surfactants, foam modulators, thickening agents, viscosity modifiers, humectants, anti-calculus or tartar control agents, sweeteners, flavorants and colorants.
1.17. Any forgoing Method, wherein the composition comprises a thickening agent, and wherein the thickening agent is selected from the group consisting of: carbomers (carboxyvinyl polymers), carrageenans, cellulosic polymers such as hydroxyethylcellulose, carboxymethylcellulose (CMC) and salts thereof, karaya, xanthan, gum arabic and tragacanth, colloidal magnesium aluminum silicate, and colloidal silica
1.18. Any foregoing Method wherein the composition is a dentifrice, e.g., a toothpaste.
1.19. Any foregoing Method wherein the composition is selected from any of Compositions 1, et seq., supra.
1.20. Any foregoing Method which is a method for reducing, inhibiting or repairing dental erosion, for example erosion of the enamel, for example wherein the composition is applied to the teeth of a patient having been identified as having dental erosion or being at elevated risk of having dental erosion.
1.21. Any foregoing Method which is a method for promoting dental remineralization, for example remineralization of the enamel, for example wherein the composition is applied to the teeth of a patient having been identified as having demineralization.
1.22. Any foregoing Method which is a method for enhancing the anti-cavity effects of fluoride, for example wherein the composition is applied to the teeth of a patient having been identified as having early signs of tooth decay, for example early enamel caries, or as having active decay, or as being at elevated risk of tooth decay.
1.23. Method 1.20 wherein the composition comprises an effective amount of fluoride and/or wherein the method further comprises administration of an oral care product comprising an effective amount of fluoride mouth rinse or toothpaste comprising an effective amount of fluoride.
1.24. Any foregoing Method, wherein the composition is free of any source of fluoride.

In another embodiment, the disclosure provides the use of a partially hydrolyzed plant protein, for example a partially hydrolyzed wheat protein or partially hydrolyzed rice protein, in the manufacture of an oral care composition, for example according to any of Compositions 1, et seq. for repairing or inhibiting dental erosion, promoting remineralization, and/or enhancing the anti-cavity effects of fluoride, e.g., in any of Methods 1, et seq.

In another aspect, the disclosure provides a method (Method 2) of making an oral care product, e.g. an oral care product useful for repairing or inhibiting dental erosion, promoting dental remineralization, and/or enhancing the anti-cavity effects of fluoride, e.g., a product according to any of Composition 1, et seq., comprising
a) a partially hydrolyzed plant protein, comprising oligo- and polypeptide molecules having a molecular weight distribution of about 500 to about 10000 D, e.g. about 1000 to about 5000 D, adjusting the pH by dilution with an aqueous buffer solution, e.g., a phosphate buffer solution, e.g., to obtain a solution having the desired pH, e.g., pH 6-8, e.g., pH 7-8;
b) filtering and centrifuging the solution product of a) to obtain a filtrate comprising the partially hydrolyzed plant protein;
c) optionally adding fluoride (e.g., in the form of an orally acceptable salt containing fluoride, for example sodium fluoride or sodium monofluorophosphate), and optionally a biocide (e.g., cetylpyridinium chloride at an effective amount, e.g., 0.01 to 1%, e.g., about 0.1% by weight of the filtrate) to the filtrate product of b);
d) admixing the product of c) to components of an orally acceptable carrier to obtain an oral care composition comprising the partially hydrolyzed plant protein (e.g., hydrolyzed wheat protein) in an amount of from 3.1 weight % to 10 weight % by total weight of the composition (e.g., about 4%, about 5%, or about 10%, by weight of the composition).
   For example, the disclosure provides:
2.1. Method 2 wherein the partially hydrolyzed plant protein is obtained from wheat, rice, almond, potato, pea, soya or combinations thereof, for example from grains of the family Poaceae, e.g., is partially hydrolyzed maize (corn), wheat, rice, barley, oat, or millet protein.
2.2. Method 2.1 wherein the partially hydrolyzed plant protein is partially hydrolyzed wheat protein or hydrolyzed rice protein.
2.3. Method 2 wherein the partially hydrolyzed plant protein is obtained from almond, potato or soya.
2.4. Any foregoing method wherein the composition is a composition according to any of Composition 1, et seq.

For example, the disclosure provides an oral care composition comprising partially hydrolyzed plant protein, e.g., a composition according to any of Composition 1, et seq., wherein the oral care composition is obtained or obtainable by the process of Method 2, et seq.

Further areas of applicability of the present invention will become apparent from the detailed description provided hereinafter. It should be understood that the detailed description and specific examples, while indicating the preferred embodiment of the invention, are intended for purposes of illustration only and are not intended to limit the scope of the invention.

### DETAILED DESCRIPTION

The following description of the preferred embodiment(s) is merely exemplary in nature and is in no way intended to limit the invention, its application, or uses.

As used throughout, ranges are used as shorthand for describing each and every value that is within the range. Any value within the range can be selected as the terminus of the range. In addition, all references cited herein are hereby incorporated by referenced in their entireties. In the event of a conflict in a definition in the present disclosure and that of a cited reference, the present disclosure controls.

Unless otherwise specified, all percentages and amounts expressed herein and elsewhere in the specification should be understood to refer to percentages by weight. The amounts given are based on the active weight of the material.

### Hydrolyzed Plant Proteins

Hydrolyzed plant proteins are proteins from plants, for example, from edible plant parts, for example from wheat, rice, almond, potato, pea, soya or combinations thereof, e.g., from cereal grains such as maize, wheat, rice, barley, oats, and millet. In particular embodiments, the hydrolyzed plant proteins are from wheat or rice.

Hydrolyzed wheat protein, also referred to as hydrolyzed wheat gluten, is typically obtained by enzymatically hydrolyzing wheat gluten using endoproteases and exoproteases. Hydrolyzed wheat protein may also be obtained through acid or alkaline hydrolysis. Methods of preparing hydrolyzed wheat protein would be known to the person skilled in the art of protein chemistry. However, hydrolyzed wheat protein is also commercially available as Gluadin® W20, Gluadin W40 from BASF, and as Wheatpro® from IKEDA. Gluadin W20 is a partial hydrolysate obtained through enzymatic hydrolysis of wheat gluten. It contains at least 20.0% of dry substance. Gluadin W40 is a partial hydrolysate obtained through enzymatic hydrolysis of wheat gluten. It contains at least 40.0% of dry substance.

In another embodiment, the hydrolyzed plant protein is made from processed wheat protein which is free of gluten.

Hydrolyzed rice protein is typically obtained by enzymatically hydrolyzing rice protein using endoproteases and exoproteases. Hydrolyzed rice protein may also be obtained through acid or alkaline hydrolysis. Methods of preparing hydrolyzed rice protein would be known to the person skilled in the art of protein chemistry. However, hydrolyzed rice protein is also commercially available as Gluadin® R from BASF, and as Rice Pro-Tein BK-S® from TRI-K Industries

The hydrolyzed plant protein used in the compositions and methods herein is not fully hydrolyzed and thus is sometimes referred to as “partially hydrolyzed” to emphasize this point. By “partially hydrolyzed” it is meant that at least some, but not all, of the peptide bonds are hydrolyzed. Thus, the hydrolyzed plant protein typically comprises a mixture of amino acids and peptides of varying size. MW of relevant active ingredient is in the range of about 500-about 10,000 D, for example about 1000 to about 5000 D.

In some embodiments, the hydrolyzed plant protein is present in the composition in an amount of from 3.1 weight % to 10 weight % by total weight of the composition. In some embodiments, the hydrolyzed plant protein is present in the composition in an amount of from 3.1 weight % to 10.5 weight % by total weight of the composition, e.g., from 3.5 weight %-10 weight % by total weight of the composition, e.g., 3.5 weight %-4.5% weight % by total weight of the composition, e.g., 4.5 weight %-5.5% weight % by total weight of the composition, e.g., 9.5 weight %-10.5% weight % by total weight of the composition, or e.g., e.g., 5.5 weight %-10.5 weight % by total weight of the composition, or about 4 weight %, or about 5 weight %, or about 6 weight %, or about 7 weight %, or about 8 weight %, or about 9 weight %, or about 10 weight % by total weight of the composition.

In one arrangement, the compositions of the present invention comprise both hydrolyzed wheat protein and hydrolyzed rice protein. In this arrangement, the hydrolyzed wheat protein and the hydrolyzed rice protein may be present in the composition in the amounts defined above. Optionally, the total amount of hydrolyzed wheat protein and hydrolyzed rice protein in the composition is from 3.1 weight % to 10 weight %, or from 3.1 weight % to 9 weight %, or from 3.1 weight % to 8 weight %, or from 3.1 weight % to 7 weight %, or from 3.1 weight % to 6 weight %, or 3.1 weight % to 5 weight % by total weight of the composition.

In some embodiments, the hydrolyzed plant protein is present in the composition in an amount of from 10.5 weight % to 30.5 weight % by total weight of the composition. In some embodiments, the hydrolyzed plant protein (e.g., hydrolyzed wheat protein) is present in the composition in an amount of about 15 weight %, or about 20 weight %, or about 25 weight %, or about 30 weight %, by total weight of the composition.

### Orally Acceptable Carrier and Optional Ingredients

The expression “orally acceptable carrier” as used herein denotes a carrier made from materials that are safe and acceptable for oral use in the amounts and concentrations intended, for example materials as would be found in conventional toothpaste and mouthwash. Such materials include water or other solvents that may contain a humectant such as glycerin, sorbitol, xylitol and the like. In some aspects, the term “orally acceptable carrier” encompasses all of the components of the oral care composition except for the hydrolyzed plant protein and the fluoride. In other aspects, the term refers to inert or inactive ingredients that serve to deliver the hydrolyzed plant protein, and/or any other functional ingredients, to the oral cavity.

Orally acceptable carriers for use in the invention include conventional and known carriers used in making mouth rinses or mouthwashes, toothpastes, tooth gels, tooth powder, lozenges, gums, beads, edible strips, tablets and the like. Carriers should be selected for compatibility with each other and with other ingredients of the composition.

The following non-limiting examples are provided. In a toothpaste composition, the carrier is typically a water/humectant system that provides a major fraction by weight of the composition. Alternatively, the carrier component of a toothpaste composition may comprise water, one or more humectants, and other functional components other than the hydrolyzed wheat protein or hydrolyzed rice protein. In a mouth rinse or a mouthwash formulation, the carrier is typically a water/alcohol liquid mixture in which the hydrolyzed wheat protein or hydrolyzed rice protein is dissolved or dispersed. In a dissolvable lozenge, the carrier typically comprises a solid matrix material that dissolves slowly in the oral cavity. In chewing gums, the carrier typically comprises a gum base, while in an edible strip, the carrier typically comprises one or more film forming polymers.

The oral care compositions provided herein may further comprise one or more additional ingredients selected from abrasives, pH modifying agents, surfactants, foam modulators, thickening agents, viscosity modifiers, humectants, anti-calculus or tartar control agents, sweeteners, flavorants, colorants and preservatives. These ingredients may also be regarded as carrier materials. Non-limiting examples are provided below.

In one embodiment a composition of the invention comprises at least one abrasive, useful, for example, as a polishing agent. Any orally acceptable abrasive can be used, but the type, fineness (particle size) and amount of abrasive should be selected so that tooth enamel is not excessively abraded during normal use of the composition. Suitable abrasives include, without limitation, silica, for example in the form of silica gel, hydrated silica or precipitated silica, alumina, insoluble phosphates, calcium carbonate, resinous abrasives such as urea-formaldehyde condensation products and the like. Among insoluble phosphates useful as abrasives are orthophosphates, polymetaphosphates and pyrophosphates. Illustrative examples are dicalcium orthophosphate dihydrate, calcium pyrophosphate, [beta]-calcium pyrophosphate, tricalcium phosphate, calcium polymetaphosphate and insoluble sodium polymetaphosphate. One or more abrasives are optionally present in the oral care compositions of the present invention in an amount of 1 weight % to 5 weight % by total weight of the composition. The average particle size of an abrasive, if present, is generally 0.1 to 30 μm, and preferably, 5 to 15 μm.

In a further embodiment an oral care composition of the invention comprises at least one bicarbonate salt, useful, for example, to impart a “clean feel” to teeth and gums due to effervescence and release of carbon dioxide. Any orally acceptable bicarbonate can be used, including, without limitation, alkali metal bicarbonates such as sodium and potassium bicarbonates, ammonium bicarbonate and the like. One or more bicarbonate salts are optionally present in a total amount of 1 weight % to 10% by weight of the composition.

In a still further embodiment, an oral care composition of the invention comprises at least one pH modifying agent. Such agents include acidifying agents to lower pH, basifying agents to raise pH and buffering agents to control pH within a desired range. For example, one or more compounds selected from acidifying, basifying and buffering agents can be included to provide a pH of 2 to 10, or in various illustrative embodiments a pH of 2 to 8, 3 to 9, 4 to 8, 5 to 7, 6 to 10, or 7 to 9. Any orally acceptable pH modifying agent can be used, including, without limitation, carboxylic, phosphoric and sulfonic acids, acid salts (for example, monosodium citrate, disodium citrate, monosodium malate), alkali metal hydroxides such as sodium hydroxide, carbonates such as sodium carbonate, bicarbonates, borates, silicates, phosphates (for example, monosodium phosphate, trisodium phosphate, pyrophosphate salts) imidazole and the like. One or more pH modifying agents are optionally present in a total amount effective to maintain the composition in an orally acceptable pH range.

In a still further embodiment a composition of the invention comprises at least one surfactant, useful, for example, to provide enhanced stability to the composition and the components contained therein, to aid in cleaning a dental surface through detergent action, and to provide foam upon agitation (for example, during brushing with a dentifrice composition of the invention). Any orally acceptable surfactant, including those which are anionic, nonionic or amphoteric, can be used. Suitable anionic surfactants include, without limitation, water-soluble salts of C₈₋₂₀ alkyl sulfates, sulfonated monoglycerides of C₈₋₂₀ fatty acids, sarcosinates, taurates and the like. Suitable nonionic surfactants include, without limitation, poloxamers, polyoxyethylene sorbitan esters, fatty alcohol ethoxylates, alkylphenol ethoxylates, tertiary amine oxides, tertiary phosphine oxides, dialkyl sulfoxides and the like. Suitable amphoteric surfactants, without limitation, derivatives of C₈₋₂₀ aliphatic secondary and tertiary amines having an anionic group such as carboxylate, sulfate, sulfonate, phosphate or phosphonate. A suitable example is cocoamidopropyl betaine. One or more surfactants are optionally present in a total amount of 0.01 weight % to 10 weight %, for example, from 0.05 weight % to 5 weight % or from 0.1 weight % to 2 weight % by total weight of the composition.

In a still further embodiment, an oral care composition of the invention comprises at least one foam modulator, useful, for example, to increase the amount, thickness or stability of foam generated by the composition upon agitation. Any orally acceptable foam modulator can be used including, without limitation, polyethylene glycols (PEGs). One or more PEGs are optionally present in a total amount of from 0.1 weight % to 10 weight by total weight of the composition.

In a still further embodiment, an oral care composition of the invention comprises at least one thickening agent, useful, for example, to impart a desired consistency and/or mouth feel to the composition. Any orally acceptable thickening agent can be used including, without limitation, carbomers (carboxyvinyl polymers), carrageenans, cellulosic polymers such as hydroxyethylcellulose, carboxymethylcellulose (CMC) and salts thereof, natural gums such as karaya, xanthan, gum arabic and tragacanth, colloidal magnesium aluminum silicate, colloidal silica and the like. One or more thickening agents are optionally present in a total amount of 0.01 weight % to 15 weight %, by total weight of the composition.

In a still further embodiment a composition of the invention comprises at least one viscosity modifier, useful, for example, to inhibit settling or separation of ingredients or to promote re-dispersion of ingredients upon agitation of a liquid composition. Any orally acceptable viscosity modifier can be used including, without limitation, mineral oil, petrolatum, clays, silica and the like. One or more viscosity modifiers are optionally present in a total amount of 0.01 weight % to 10 weight %, by total weight of the composition.

In a still further embodiment, an oral care composition of the invention comprises at least one humectant which may be used to prevent hardening of a toothpaste upon exposure to air. Any orally acceptable humectant can be used, including, without limitation, polyhydric alcohols such as glycerin, sorbitol, xylitol or low molecular weight PEGs. Most humectants also function as sweeteners. One or more humectants are optionally present in a total amount of 1 weight % to 50 weight % by total weight of the composition.

In a still further embodiment, an oral care composition of the invention comprises at least one sweetener which enhances taste of the composition. Any orally acceptable natural or artificial sweetener can be used including, without limitation, dextrose, sucrose, maltose, dextrin, mannose, xylose, ribose, fructose, levulose, galactose, corn syrup, partially hydrolyzed starch, hydrogenated starch hydrolysate, sorbitol, mannitol, xylitol, maltitol, isomalt, aspartame, neotame, saccharin and salts thereof, dipeptide-based intense sweeteners, cyclamates and the like. One or more sweeteners are optionally present in a total amount of 0.005 weight % to 5 weight % by total weight of the composition.

In a still further embodiment, an oral care composition of the invention comprises at least one flavorant which enhances the taste of the composition. Any orally acceptable natural or synthetic flavorant can be used including, without limitation, vanillin, sage, marjoram, parsley oil, spearmint oil, cinnamon oil, oil of wintergreen (methylsalicylate), peppermint oil, clove oil, bay oil, anise oil, eucalyptus oil, citrus oils, fruit oils and essences, and the like. Also encompassed within flavorants are ingredients that provide fragrance and/or other sensory effects in the mouth, including cooling or warming effects. Such ingredients illustratively include menthol, menthyl acetate, menthyl lactate, camphor, eucalyptus oil, eucalyptol, eugenol, cassia, oxanone, α-irisone, thymol, linalool, benzaldehyde, cinnamaldehyde, N-ethyl-p-menthan-3-carboxamine, N,2,3-trimethyl-2-isopropylbutanamide, 3-(1-menthoxy)-propane-1,2-diol, cinnamaldehyde glycerol acetal (CGA), menthone glycerol acetal (MGA) and the like. One or more flavorants are optionally present in a total amount of 0.01 weight % to 5 weight %, by total weight of the composition.

In a still further embodiment, an oral care composition of the invention comprises at least one colorant. A colorant can serve a number of functions. These include providing a white or light-colored coating on a dental surface, indicating locations on a dental surface that have been effectively contacted by the composition, and/or modifying the appearance of the composition to enhance attractiveness to the consumer. Any orally acceptable colorant can be used including, without limitation, talc, mica, magnesium carbonate, calcium carbonate, magnesium silicate, magnesium aluminum silicate, silica, titanium dioxide, zinc oxide, iron oxide, ferric ammonium ferrocyanide, manganese violet, titaniated mica, bismuth oxychloride and the like. One or more colorants are optionally present in a total amount of 0.001 weight % to 20 weight % by total weight of the composition.

In a still further embodiment, an oral care composition of the invention comprises a preservative. The preservative may be selected from parabens, potassium sorbate, benzyl alcohol, phenoxyethanol, polyaminopropryl biguanide, caprylic acid, sodium benzoate and cetylpyridinium chloride. In some embodiments, the preservative is present at a concentration of from about 0.001 to about 1 weight %, by total weight of the composition.

The following examples illustrate compositions of the invention and their uses. The exemplified compositions are illustrative and do not limit the scope of the invention.

### EXAMPLES

### Example 1—Microhardness

Bovine teeth are cut, ground and polished to obtain enamel blocks having approximate dimensions of 3 mm×3 mm×2 mm. The thickness of the enamel is approximately 1 to 2 mm, and the thickness of dentin is approximately 1 mm. All measurements are taken on the enamel surface.

Microhardness is measured using a Micromet 6020 Micro-hardness Tester with a Knoop Diamond Indenter and a 50 g load (Buehler, Lake Bluff, Ill., USA). Blocks with a Knoop hardness (KH) of at least 300 are selected. The blocks are etched by immersing in 30% phosphoric acid for 15 seconds. Then blocks are washed with DI water @5 minutes, 500 RPM and air dried overnight. Microhardness for each etched block is measured. Subsequently, the blocks are treated with 2 ml of corresponding weight concentration solution of hydrolyzed wheat protein (Gluadin W20 from BASF), for 30 minutes. The blocks are washed twice with DI water @ 5 minutes, 500 PRM after the treatment. Subsequently, the blocks are incubated in AS solution (0.2 mM MgCl₂, 1 mM CaCl₂).H₂O, 20 mM HEPES buffer, 4 mM KH₂PO₄, 16 mM KCl, 4.5 mM NH₄Cl at pH 7 (adjusted with 1M NaOH)) overnight. After rinsing the enamel blocks with DI water and air-drying the rinsed blocks, microhardness is measured again. Surface microhardness regain (SMHL, Remin %) as a percent is calculated as

$$\% remineralization = \frac{( {MH}_{Repaired} - {MH}_{Etched} )}{( {MH}_{Baseline} - {MH}_{Etched} )} \times 100$$

The results of the microhardness assay are illustrated in Table 1.

TABLE 1
Results of microhardness assay

| Protein | % remineralization (mean) | S.D. |
|---|---|---|
| 3% Hydrolyzed wheat protein | 27.8% | 5.9% |
| 4% Hydrolyzed wheat protein | 38.8% | 8.2% |
| 5% Hydrolyzed wheat protein | 42.0% | 0.7% |
| 10% Hydrolyzed wheat protein | 49.4% | 5.8% |
| Control (0% Hydrolyzed wheat | 9.32% | 6.37% |
| protein) | | |

It can be seen from Table 1 that hydrolyzed wheat protein is effective in remineralizing the enamel surface of acid-etched enamel blocks. In particular, higher concentrations of hydrolyzed wheat protein appear to increase the remineralization effect. The only differences in the formulations are the amounts (or presence) of hydrolyzed wheat protein. The test formulations in Table 1 as simple solutions (using deionized water) corresponding to the weight percentages of hydrolyzed wheat protein.

Formulations with the hydrolyzed wheat protein are also effective in remineralizing the enamel surface of acid-etched enamel blocks when compared to oral care composition formulations containing fluoride. The results of such a comparison, using the microhardness assay illustrated above, is illustrated in Table 2.

TABLE 2
Results of microhardness assay (Comparison
w./Fluoride formulations)

| Protein | % remineralization (mean) | S.D. |
|---|---|---|
| 4% Hydrolyzed wheat protein | 42.1 | 3.6% |
| 225 ppm F | 26.0% | 2.8% |
| Control - Water (0% | 11.4% | 3.94% |
| Hydrolyzed wheat protein) | | |

### Example 2

An exemplary gel formulation (e.g., tooth gel) is prepared comprising the following ingredients:

| Ingredients | Amounts (by wt %) |
|---|---|
| Demineralized water | q.s. |
| Poloxamer 338 | 0.5 |
| Sodium Saccharin | 0.02 |
| Hydrolyzed Wheat Protein | 4.0 |
| Cetylpyridinium Chloride | 0.04 |
| Potassium Sorbate | 0.05 |
| Glycerin - USP, EP VEG | 7.5 |
| Sorbitol - Non Crystal - | 5.5 |
| 70% Solution USP, EP | |
| Propylene Glycol | 7 |
| Benzyl Alcohol | 0.20 |
| Xanthan Gum | 1.6 |
| Flavoring | 0.14 |
| Total Components | 100% |

### Example 3

An exemplary gel formulation (e.g., tooth gel) is prepared comprising the following ingredients:

| Ingredients | Amounts (by wt %) |
|---|---|
| Demineralized water | q.s. |
| Poloxamer 338 | 0.5 |
| Sodium Saccharin | 0.02 |
| Hydrolyzed Wheat Protein | 4.0 |
| Cetylpyridinium Chloride | 0.04 |
| Potassium Sorbate | 0.05 |
| Glycerin - USP, EP VEG | 7.5 |
| Sorbitol - Non Crystal - | 5.5 |
| 70% Solution USP, EP | |
| Propylene Glycol | 7 |
| Benzyl Alcohol | 0.20 |
| Hydroxyethylcellulose | 4.0 |
| Flavoring | 0.14 |
| Total Components | 100% |

The above exemplary formulation provides a fluoride-free alternative oral gel. For example, the above formulation can be used as an in-office dental preparation.

While particular embodiments of the invention have been illustrated and described, it will be obvious to those skilled in the art that various changes and modifications may be made without departing from the scope of the invention as defined in the appended claims.

1. An oral care composition comprising:
a. partially hydrolyzed plant protein, wherein the partially hydrolyzed plant protein comprises oligo- and polypeptide molecules having a molecular weight distribution of about 500 D to about 10000 D; and
b. an orally acceptable carrier,
wherein the partially hydrolyzed plant protein is present in the composition in an amount of from 3.05 weight % to 10.5 weight % by total weight of the composition.

2. The oral care composition of claim 1 wherein the partially hydrolyzed plant protein is obtained from grains of the family Poaceae.

3. The oral care composition of claim 1, wherein the partially hydrolyzed plant protein is partially hydrolyzed wheat protein or hydrolyzed rice protein.

4. The oral care composition of claim 1, wherein the partially hydrolyzed plant protein is obtained from wheat, rice, almond, potato, pea, soya or combinations thereof.

5. The oral care composition of claim 1, wherein the oral care composition comprises fluoride.

6. The oral care composition of claim 1, wherein the composition is free of any fluoride source.

7. The oral care composition of claim 1, wherein the partially hydrolyzed plant protein is present in the composition in an amount of from 3.5 weight %-10.5 weight % by total weight of the composition.

8. The oral care composition of claim 1, wherein the partially hydrolyzed plant protein is present in the composition in an amount of from 3.05 weight % to 30.5 weight % by total weight of the composition.

9. The oral care composition of claim 1, wherein the partially hydrolyzed plant protein is present in the composition from 3.5 weight %-4.5% weight % by total weight of the composition.

10. The oral care composition of claim 1, wherein the composition is in a form selected from a mouthrinse, a toothpaste, a tooth gel, a tooth powder, a non-abrasive gel, a mousse, a foam, a mouth spray, chewing gum, and a tablet.

11. The oral care composition of claim 1, wherein the composition is in the form of a gel.

12. A method of repairing or inhibiting dental erosion, promoting dental remineralization, and/or enhancing the anti-cavity effects of fluoride comprising applying to the teeth a composition comprising:
a) partially hydrolyzed plant protein, comprising oligo- and polypeptide molecules having a molecular weight distribution of about 500 to about 10000 D;
b) an orally acceptable carrier,
wherein the partially hydrolyzed plant protein is present in the composition in an amount of from 3.5 weight %-10 weight % by total weight of the composition.

13. The method of claim 12, wherein the partially hydrolyzed plant protein is partially hydrolyzed wheat protein or hydrolyzed rice protein.

14. The method of claim 12, wherein the partially hydrolyzed plant protein is obtained from almond, potato or soya.

15. The method of claim 12, wherein the oral care composition comprises fluoride.

16. The method of claim 12, wherein the oral composition is free of any source of fluoride.

17. A method of making an oral care product useful for repairing or inhibiting dental erosion, promoting dental remineralization, and/or enhancing the anti-cavity effects of fluoride, comprising:
a) a partially hydrolyzed plant protein, comprising oligo- and polypeptide molecules having a molecular weight distribution of about 500 to about 10000 D, adjusting the pH by dilution with an aqueous buffer solution, to obtain a solution having the desired pH;
b) filtering and centrifuging the solution product of a) to obtain a filtrate comprising the partially hydrolyzed plant protein;
c) optionally adding fluoride and optionally a biocide;
d) admixing the product of c) to components of an orally acceptable carrier to obtain an oral care composition comprising the partially hydrolyzed plant protein in an amount of from 3.1 weight % to 10 weight % by total weight of the composition.
